(19) United States
(12) Patent Application Publication
(10) Pub. No.: US 2023/0201123 A1
(43) Pub. Date: Jun. 29, 2023

(54) PHARMACEUTICAL COMPOSITION

(71) Applicant: GLAXOSMITHKLINE CONSUMER HEALTHCARE HOLDINGS (US) LLC, Wilmington, DE (US)

(72) Inventors: Shripad JATHAR, Telengana (IN); Khan Mohammed MAJED, Telengana (IN)

(21) Appl. No.: 17/998,863

(22) PCT Filed: May 6, 2021
(86) PCT No.: PCT/US2021/031016

§ 371 Date: Nov. 15, 2022

(30) Foreign Application Priority Data
May 22, 2020 (GB) 2007670.9

(51) Int. Cl. A61K 9/20; A61K 31/167; A61K 31/616; A61K 31/522

(52) U.S. Cl. CPC A61K 9/2059; A61K 9/2009; A61K 9/2013; A61K 9/2027; A61K 31/167; A61K 31/522; A61K 31/616

(57) ABSTRACT

A pharmaceutical composition, suitably in the form of a tablet, is described comprising intragranular and extragranular components. The intragranular components comprise a pharmaceutically effective amount of paracetamol, calcium carbonate provided in the form of particles having a d50 greater than 11 µm, and at least one binding agent and the extragranular components comprise a pharmaceutically effective amount of aspirin, and at least one hydrophilic colloid. Optionally the compositions comprise caffeine.

### INVENTION FIELD

The present invention relates generally to pharmaceutical compositions comprising aspirin, paracetamol and optionally a paracetamol adjuvant such as caffeine. More particularly, the present invention relates to a composition that aims to provide a fast and/or enhanced release of its active components. In one aspect the invention further provides a composition that demonstrates acceptable long-term storage stability.

### BACKGROUND

Aspirin, also known as acetylsalicylic acid (ASA), is a well-known non-steroidal antiinflammatory drug (NSAID) and antipyretic active ingredient. It has been known to combine aspirin with other pharmaceutically active agent(s) such as the analgesic and antipyretic agent, paracetamol (also known as N-acetyl-p-aminophenol, acetaminophen and APAP), to provide a dual combination analgesic/antipyretic product.

Examples of commercially available combination analgesic/antipyretic products containing aspirin and paracetamol and sometimes caffeine), are marketed under the tradename EXCEDRIN, by GlaxoSmithKline Consumer Health. Such products, containing a triple combination of aspirin, paracetamol and caffeine, have been shown to provide superior efficacy and speed of onset as compared to formulations containing a single NSAID alone (Goldstein J., Silberstein S. et al., Headache 46(3), 2006, p. 444 - 453, doi: 10.1111/j.1526-4610.2006.00376.x).

WO2006/049978 (Novartis AG) discloses compositions comprising acetaminophen, caffeine, aspirin and an alkaline agent for enhanced absorption. According to WO2006/049978, inclusion of a buffer/alkaline species, for example a carbonate, a bicarbonate or a mixture thereof, optionally with a pharmaceutically acceptable magnesium salt, in formulations containing analgesic/antipyretic actives, such as aspirin and acetaminophen and combinations of aspirin and/or acetaminophen, with caffeine, enhances the dissolution rate, speeds gastric emptying time, and possibly stimulates the gastro-intestinal tract, so that the analgesic/antipyretic actives are more rapidly absorbed and the onset of the analgesic/antipyretic effect is shortened. Further, according to WO2006/049978, an alkaline agent/antacid, for example a carbonate or bicarbonate salt, an oxide or a hydroxide, in a composition comprising paracetamol, aspirin and caffeine, accelerates the plasma uptake of paracetamol and aspirin whilst being bioequivalent to a composition without the alkaline agent/antacid. WO2006/049978 also refers to the prior art suggesting that for paracetamol, a faster rate of absorption entails the clinical benefit of a faster onset of analgesic action and possibly a greater peak analgesic effect.

As acknowledged in WO2006/049978, the alkaline agent must be prevented from reacting with the acidic analgesic/antipyretic active ingredients to ensure stability of the formulation. The unit dosage forms described therein include a powder which can be packaged in a double pouch, in order to separate the acidic analgesic/antipyretic component(s) and alkaline agent(s). Alternatively, the alkaline agent(s) can be contained in one tablet or caplet and the analgesic/antipyretic component(s) can be contained in a separate tablet or caplet. In one embodiment, the aspirin and acetaminophen (paracetamol) are formulated in one layer of a multi-layer tablet or caplet and the alkaline agent(s) is (are) formulated in another layer of the multi-layer tablet or caplet. Another means suggested of keeping the acidic analgesic/antipyretic component(s) of the composition from interacting with the alkaline agent(s) is by placing the acidic analgesic/ antipyretic component(s) in a capsule and inserting the capsule into another capsule containing the alkaline agent(s). Alternatively, the alkaline agent(s) can be placed in a capsule and the capsule can be inserted into another capsule containing the acidic analgesic/antipyretic component(s). The caffeine can be present in either the inner or outer capsule or apportioned between both capsules. Instead of a capsule within another capsule, the dosage form can be a tablet within a capsule or a tablet compressed within another tablet. Yet another means described of keeping the acidic analgesic/antipyretic component(s) of the composition from interacting with the alkaline agent(s) is coating the alkaline agent(s), the acidic analgesic/antipyretic component(s) or both, so as to create a physical barrier between the two that prevents their interaction.

The dosage forms proposed by WO2006/049978 are complex thus leading to a likelihood of higher manufacturing costs and are of potential inconvenience for the patient resulting in decreased compliance and general dissatisfaction with the product.

WO2007/118063 relates to fast release paracetamol products and discloses pharmaceutical compositions comprising paracetamol, calcium carbonate (CaCO₃) and at least one binding agent and at least one disintegrating agent as intragranular components in the form of a granulate. The compositions satisfy the requirements of a specified dissolution test with respect to release rate of paracetamol. The compositions are formulated so as to disintegrate and dissolve rapidly in the stomach thereby facilitating fast absorption of paracetamol into the circulatory system. Whilst WO2007/118063 generally contemplates a formulation comprising aspirin, there is no specific disclosure of how to formulate a composition comprising aspirin, paracetamol and calcium carbonate.

One of the challenges associated with the formulation of aspirin is the provision of a product that is stable. Aspirin is susceptible to hydrolytic degradation resulting in the formation of free salicylic acid and acetic acid. The hydrolytic cleavage of aspirin to salicylic acid and acetic acid is catalysed by alkali metal ions and alkaline earth metal ions such as the cations of calcium carbonate, sodium carbonate and sodium bicarbonate:

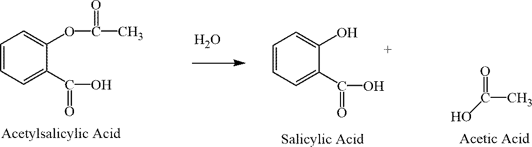

Salicylic acid is recognized as an impurity in pharmaceutical products containing aspirin and its presence above a certain level in such products is not acceptable. The United States Pharmacopoeia (USP) monograph for tablets comprising paracetamol, aspirin and caffeine has, as an acceptance criterion, a limitation on the amount of free salicylic acid being not more than 3% the amount of aspirin in a tablet (see Second Supplement to USP 40-NF 35). The second resulting degradation product, acetic acid, additionally has a distinct and unpleasant smell and taste that is not desirable for a pharmaceutical product, especially one intended for oral intake.

There is a continuing need in the art for oral dosage forms of paracetamol and aspirin-containing compositions, optionally comprising caffeine, that provide a rapid attainment of maximum blood concentration of the active components (paracetamol and aspirin), along with a greater prospect for rapid onset of therapeutic effect. In a further aspect there remains a need to provide compositions that are stable.

### SUMMARY

Aspects of the present invention provide a pharmaceutical composition, suitably in the form of a swallow tablet, comprising particular intragranular and extragranular components. It has now been found, unexpectedly, that by formulating paracetamol with a binding agent and calcium carbonate of a particular particle size, as intragranular components, and aspirin and a hydrophilic colloid, as extragranular components, a composition can be provided that demonstrates desirable characteristics, such as improved dissolution properties. A composition according to the invention is economical to manufacture and is sufficiently robust so as to be capable of withstanding packaging, shipping and handling operations. A composition according to the invention can be administered orally as a compressed tablet or as free-flowing particles encapsulated in a hard shell, for example a gelatin shell. When in the form of a tablet, such as a swallow tablet, the tablet may be coated or uncoated.

Advantageously, unlike compositions disclosed in WO2006/049978, there is no requirement in a composition according to the present invention to physically separate the calcium carbonate (the alkaline agent) from the aspirin (the acidic analgesic/antipyretic component).

In one aspect the present invention provides a pharmaceutical composition comprising intragranular and extragranular components wherein the intragranular components comprise a pharmaceutically effective amount of paracetamol, calcium carbonate provided in the form of particles having a d50 greater than 11 µm and at least one binding agent, and wherein the extragranular components comprise a pharmaceutically effective amount of aspirin, and at least one hydrophilic colloid.

In one aspect the present invention provides a pharmaceutical composition in the form of a tablet comprising intragranular and extragranular components wherein the intragranular components comprise a pharmaceutically effective amount of paracetamol, calcium carbonate provided in the form of particles having a d50 greater than 11 µm, and at least one binding agent, and wherein the extragranular components comprise a pharmaceutically effective amount of aspirin, and at least one hydrophilic colloid. The tablet may optionally comprise a film coat.

Aspects of the present invention further relate to the use of such compositions for the treatment of pain.

### BRIEF DESCRIPTION OF THE DRAWINGS

FIG. 1: Active pharmaceutical ingredient (API) excipient compatibility study

FIG. 2: Effect of particle size of calcium carbonate and grade of aspirin or salicylic acid formation under long-term study conditions (25° C., 60% RH)

FIG. 3: Effect of particle size of calcium carbonate and grade of aspirin on salicylic acid formation under accelerated study conditions (40° C., 75% RH)

FIG. 4: Comparative dissolution study

### DETAILED DESCRIPTION

For purposes herein, a reference to a composition that is “stable” means a composition that demonstrates pharmaceutically acceptable storage stability, suitably commensurate with that required for a commercial product having a desired shelf-life, typically of at least one or two years, in one aspect the present invention provides a composition wherein the amount of free salicylic acid does not exceed 3% of the initial amount of aspirin in the composition following storage at 40° C. (°C) and 75% relative humidity (RH) for six months.

Dissolution as used herein is determined by measurement of the percentage of a component that is found in solution after a specified time under specified dissolution conditions.

“Specified dissolution conditions” refers to a dissolution testing method which uses a USP paddle apparatus rotating at 30 rotations per minute (RPM), employing 900 ml of 0.05 M HCI at 37° C. as dissolution medium. The percentage amount of compound dissolved in the dissolution medium is determined at six minutes as the specified time.

“Dissolution time” is the time needed for dissolution of a component in a dissolution medium.

For purposes herein, “fast release” means that at least 60% by weight, for example at least 70% by weight or at least 80% by weight, of a compound, is dissolved from a composition under specified dissolution conditions.

For purposes herein “enhanced release”, means that a higher percentage amount of a compound is dissolved from a composition according to the invention under specified dissolution conditions. For purposes herein, “enhanced release” means that at least 2 times, for example at least 3 times or at least 4 times the amount of a compound is dissolved from a composition within a specified time frame under specified dissolution conditions, as compared to a control composition under the same conditions. In one embodiment the compound is aspirin. The control composition comprises the same amount of aspirin but does not comprise calcium carbonate and a hydrophilic colloid.

For purposes herein, a component is referred to as “intragranular”, “present intragranularly” or as “intragranular component” when used in the pharmaceutical composition in the form of a granulate.

A granulate for use in the present invention is prepared by subjecting intragranular components to a granulation method, for example as described in WO2007/118063.

Well-known granulation methods include wet granulation and dry granulation. In one embodiment the intragranular components are granulated by wet granulation. Wet granulation comprises the steps of mixing of the dry intragranular components, contacting the thus obtained mixture with a granulation binder to yield a granulate, forming of granules from the granulate and drying of the thus obtained wet granules to obtain a granulate of a desired residual moisture content. It is apparent that in the embodiments employing wet granulation as the granulation method, a certain residual content of the granulation binder is also present as an intragranular component, and thus in the total composition. Typically, the granulation binder is a solvent such as water, an aqueous solvent or an organic solvent. In the case of aqueous wet granulation, the granulation binder is water or an aqueous solvent. In one embodiment, the intragranular components are granulated by aqueous wet granulation. Surprisingly, it has been found that although residual water may be present in some embodiments of the invention, the stability of aspirin (present extragranularly) is not compromised. Drying of granules and granulates to a desired residual moisture content may be achieved by any drying technique known in the art, for example drying in a batch drying oven, a continuous drying oven or a fluid bed dryer. To achieve a uniform particle size distribution of the granulate, the granulate may be sifted through sieves of a suitable mesh size and the retention may be milled and sieved again. The granules thus obtained may subsequently be processed further.

According to the invention, the intragranular components comprise a pharmaceutically effective amount of paracetamol, calcium carbonate provided in the form of particles having a d50 greater than 11 µm, and at least one binding agent The intragranular components may further comprise one or more additional components such as one or more additional active pharmaceutical ingredients (API) and pharmaceutically acceptable excipients, such as a filler, a bulking agent, a disintegrating agent, a diluent, a sweetening agent, a lubricant, a glidant, a flow aid, a compression aid, a colorant, a preservative, a wetting agent, an adhesive or a solvent.

For purposes herein a component is referred to as “extragranular”, “present extragranularly” or as “extragranular component” when it is present in the pharmaceutical composition extrinsic to said granulate, Extragranular components are admixed with the granulate in the process of forming the pharmaceutical compositions of the invention.

According to the invention, the extragranular components comprise a pharmaceutically effective amount of aspirin, at least one hydrophilic colloid, and, optionally, caffeine. The extragranular components may further comprise one or more additional components such as one or more additional APIs and pharmaceutically acceptable excipients, such as a filler, a bulking agent, a binding agent, a disintegrating agent, a diluent, a sweetening agent, a lubricant, a glidant, a flow aid, a compression aid, a colorant, a preservative, a wetting agent, an adhesive or a solvent.

### Paracetamol

A pharmaceutical composition according to the invention comprises a pharmaceutically effective amount of paracetamol as an intragranular component. A “pharmaceutically effective amount” is to be understood as an amount of an API, such as paracetamol, that is able to achieve a biological response, i.e. a pharmacological effect in a human subject. The amount of API needed to achieve such an effect varies for different APIs and also varies between individual human subjects. Even in one human subject, the amount needed to achieve a pharmacological effect may vary at different points in time, for example between the fed and the fasted state. Depending on the human subject, the amount needed to achieve a pharmacological effect can be higher, for example for adults, larger people, people with acute symptoms or when a short-term intake is envisaged, or lower, for example for children, elderly people or when long-term intake is envisaged.

Suitably the pharmaceutically effective amount of paracetamol is an amount of 100 mg to 1000 mg per unit dosage form (e.g. per tablet), such as 50 mg to 500 mg, for example 250 mg. Suitably the paracetamol is present in a composition according to the invention in an amount up to 50% by weight of the composition. In one embodiment, the paracetamol is present in the pharmaceutical composition in an amount from 20% to 50% by weight of the composition. In one embodiment, the paracetamol is present in the pharmaceutical composition in an amount from 25% to 45% by weight of the composition. In one embodiment, the paracetamol is present in the pharmaceutical composition in an amount from 30% to 40% by weight of the composition. In one embodiment, the paracetamol is present in the pharmaceutical composition in an amount from 31% to 38% by weight of the composition.

### Paracetamol Adjuvant

In one aspect a pharmaceutical composition according to the invention further comprises a paracetamol adjuvant. The paracetamol adjuvant may be present as an intragranular or extragranular component. In one embodiment the paracetamol adjuvant is caffeine. In one embodiment, caffeine is present as an extragranular component. Suitably the paracetamol adjuvant, such as caffeine, is present in an amount of 15 mg to 250 mg per unit dosage form (e.g. per tablet), such as 30 mg to 150 mg, for example 65 mg.

In one embodiment the paracetamol adjuvant, such as caffeine, is present in an amount from 1% to 20% for example from 4% to 15% by weight of the composition. In one embodiment, paracetamol adjuvant, such as caffeine, is present in an amount from 7% to 11% by weight of the composition. In one embodiment, the extragranular components comprise a blend of caffeine, aspirin and pregelatinized starch.

### Aspirin

A pharmaceutical composition according to the invention comprises a pharmaceutically acceptable grade of aspirin, present as an extragranular component. Commercially available grades of aspirin are generally coarse, and their particle size is suitably characterized by mesh size, meaning that the aspirin is screened through sieves having a particular mesh size so that only particles having at least one diameter smaller than this mesh size pass through the sieve. The determined “mesh”, or “mesh size”, is a term used in the art to characterize the particle size of coarse or granular products with a nonuniform shape. The values obtained may, for example, be expressed in “standard mesh” or “sieve number”. As referred to herein, mesh number refers to the U.S. standard mesh numbers. A product characterized by “mesh number” or “mesh 30”, for example, contains only particles that have passed a U.S. standard mesh 30, which is defined to have openings of 600 µm.

A fine grade of particulate components, corresponding to smaller particles, is generally preferred for use in fast dissolving tablets since such tablets generally show better wetting properties resulting in faster disintegration and dissolution times. However the present inventors discovered that use of a fine grade of aspirin may not be desirable when formulating a tablet composition comprising aspirin, calcium carbonate and paracetamol. The present inventors found that a fine grade of aspirin resulted in a modest increase in degradation of aspirin, whereas, the same was not found when a coarser grade of aspirin, for example characterized by a mesh number of 20, 30 or 40, was used.

In one embodiment, a composition of the invention comprises aspirin having a particle size of 20 to 40 mesh, for example 30 to 40 mesh such as 40 mesh. Mesh size may be correlated with the following particle size distributions:

| Mesh Size (USP standard mesh) | Particle size distribution (PSD) |
|---|---|
| 20 | At least 70% particles have a PSD in the range 180 µm - 850 µm |
| 30 | At least 60% particles have a PSD in the range 420 µm - 850 µm |
| 40 | At least 85% particles have a PSD in the range 105 µm - 420 µm |

Accordingly in one embodiment the aspirin for use in the invention is provided in particulate form wherein at least 70% of the aspirin particles have a particle size distribution in the range 180 µm to 850 µm. In another embodiment, the aspirin for use in the invention is provided in particulate form wherein at least 60% of the aspirin particles have a particle size distribution in the range are 420 µm to 850 µm. In a further embodiment the aspirin for use in the invention is provided in particulate form wherein at least 85% particles have a particle size distribution in the range 105 µm to 420 µm.

Suitably, the pharmaceutically effective amount of aspirin of use in the invention is an amount of 25 mg to 2500 mg per unit dosage form (e.g. per tablet), such as from 150 mg to 500 mg, for example 250 mg. In one embodiment, the aspirin is present in the pharmaceutical composition in an amount from 10% to 60% by weight of the composition. In one embodiment, the aspirin is present in the pharmaceutical composition in an amount from 20% to 50% by weight of the composition. In one embodiment, the aspirin is present in the pharmaceutical composition in an amount from 30% to 40% by weight of the composition. In one embodiment, the aspirin is present in the pharmaceutical composition in an amount from 32% to 38% by weight of the composition.

### Calcium Carbonate

A pharmaceutical composition according to the present invention comprises calcium carbonate of a particular particle size distribution as an intragranular component. Suitably the calcium carbonate of use in the invention is precipitated calcium carbonate.

One of the most widely used methods of describing particle size distribution is via percentiles or “d” values. The d10, d50 and d90 values are particle size values corresponding to the cumulative distribution at 10%, 50% and 90%. Depending on the method used, the distribution is based on number, mass or volume.

As used herein, the term “d10” refers to a size in microns below which 10% of the particles reside on a volume basis.

As used herein, “d50”, also known as the median value or median diameter, refers to a size in microns above or below which 50% of the particles reside on a volume basis.

As used herein, the term “d90” refers to a size in microns below which 90% of the particles reside on a volume basis.

In the present invention the d10, d50 and d90 values are reported as a volume based particle diameter measured by a laser diffraction method, for example using a laser diffraction particle size analyser such as the Malvern Mastersizer 2000. It is to be understood that the particle size values of calcium carbonate refer to particle size prior to formulation with other components of a composition according to the invention.

Calcium carbonate of use herein, has a d50 of greater than 11 µm. In one embodiment, the calcium carbonate has a d50 from 11 µm to 30 µm or from 12 µm to 25 µm. In one embodiment, the calcium carbonate has a d50 of 14 µm to 20 µm. In a further embodiment, the calcium carbonate has a d50 of 15 µm to 18 µm.

Suitably the calcium carbonate particles have a particle size distribution range having a d50 greater than 11 µm and a d90 less than 60 µm, for example less than 50 µm or less than 45 µm. In one embodiment, the calcium carbonate particles have a d50 from 11 µm to 30 µm and a d90 is less than 60 µm, for example less than 50 µm or less than 45 µm. In one embodiment, the calcium carbonate particles have a d50 from 12 µm to 25 µm and a d90 less than 60 µm, for example less than 50 µm or less than 45 µm. In one embodiment, the calcium carbonate particles have a d50 of 14 µm to 20 µm and a d90 less than 60 µm, for example less than 50 µm or less than 45 µm. In a further embodiment, the calcium carbonate particles have a d50 of 15 µm to 18 µm and a d90 less than 60 µm, for example less than 50 µm or less than 45 µm.

In one embodiment, the calcium carbonate particles have a d50 from 11 µm to 30 µm and a d90 less than 60 µm, for example less than 50 µm or less than 45 µm and a d10 less than 10 µm, for example less than 5 µm. In one embodiment, the calcium carbonate particles have a d50 from 12 µm to 25 µm and a d90 less than 60 µm, for example less than 50 µm or less than 45 µm, and a d10 less than 10 µm, for example less than 5 µm. In one embodiment, the calcium carbonate particles have a d50 of 14 µm to 20 µm and a d90 less than 60 µm, for example less than 50 µm or less than 45 µm and a d10 less than 10 µm, for example less than 5 µm. In a further embodiment, the calcium carbonate particles have a d50 of 15 µm to 18 µm and a d90 less than 60 µm, for example less than 50 µm or less than 45 µm and a d10 less than 10 µm, for example less than 5 µm.

Without being bound to a specific theory, it is believed that in the acidic environment of the stomach, the intragranular calcium carbonate reacts with the extragranular hydrophilic colloid, and, in combination with carbon dioxide, generated from the calcium carbonate, facilitates the formation of a suspension of fine particles with increased surface area, leading to enhanced dissolution.

Under the specified dissolution conditions used herein, the dissolution characteristics of aspirin and paracetamol and caffeine in a composition according to the invention are improved relative to a control composition.

In one embodiment a composition according to the invention demonstrates enhanced release of at least one of aspirin, paracetamol and caffeine. In one embodiment a composition according to the invention demonstrates a fast release of at least one of paracetamol and caffeine.

Suitably, calcium carbonate is present in an amount from 0.5% to 10%, such as from 1% to 8%, by weight of the composition. In one embodiment, the calcium carbonate is present in an amount up to 6% by weight of the composition for example in an amount ranging from 2% to 5% by weight of the composition. Surprisingly, incorporation of just a small amount of calcium carbonate of an appropriate particle size can afford an improvement in dissolution characteristics of paracetamol and aspirin, as compared to a composition containing no calcium carbonate.

However as known in the art, co-formulation of calcium carbonate with aspirin can adversely affect stability of the aspirin. The present inventors have surprisingly found that stability of aspirin can be improved by using calcium carbonate having a d50 greater than 11 µm as an intragranular component and by using aspirin as an extragranular component. The effect of different grades of aspirin (coarse versus fine) did not outweigh the increased degradation of aspirin observed in compositions comprising calcium carbonate having a d50 smaller than 11 µm.

### Binding Agent

A pharmaceutical composition according to the invention comprises at least one binding agent. In one embodiment the binding agent(s) are present solely intragranularly. In one embodiment the binding agent(s) are present intragranularly and extragranularly.

Suitable binding agents include conventional binding agents used in the art for example as described in WO 2007/118063. In one embodiment the binding agent is a starch, polymer, cellulose derivative or a combination of two or more thereof. Suitably the binding agent(s) are present in amount from 0.5% to 20%, for example from 1 to 15% by weight of the composition.

Examples of suitable starches include corn (or maize) starch, modified corn starch, wheat starch, modified wheat starch, potato starch, or pregelatinized starch e.g. available commercially as Starch 1500G or Prejel; or a combination of two or more thereof. In one embodiment the binding agent comprises pregelatinized starch.

Pregelatinized starch is a starch that has been chemically and/or mechanically processed. Typically, pregelatinized starch contains 5% of free amylase, 15% of free amylopectin and 80% unmodified starch. Pregelatinized starch may be obtained from corn (or maize), potato or rice starch.

In one embodiment the binding agent comprises pregelatinized starch, present in an amount from 1% to 20% for example 5% to 15%, by weight of the composition. In one embodiment, the binding agent comprises pregelatinized starch present intragranularly in an amount from 1% to 10%, for example from 3% to 8%, by weight of the composition, and extragranularly in an amount from 1% to 10%, for example from 4% to 8%, by weight of the composition.

In one embodiment the binding agent comprises a polymer, selected from povidone (PVP), polyvinyl alcohol (PVA), polyethylene oxide, poloxamer, polymethacrylate, a carbomer, polyethylene glycol and calcium polycarbophil, or a combination of two or more thereof. In one embodiment the polymer comprises povidone. In one embodiment the povidone is present intragranularly. Suitably the povidone comprises an approximate molecular weight of 30,000.

Suitably when present the polymer is present in an amount from 0.01% to 5% for example from 0.05% to 0.5% by weight of the composition. In one embodiment the polymer comprises povidone present intragranularly in an amount from 0.01% to 5% by weight of the composition.

Examples of suitable cellulose derivatives comprise hydroxypropyl cellulose (HPC), hydroxypropyl methyl cellulose (HPMC), microcrystalline cellulose (MCC), carboxymethylcellulose (MC), sodium carboxymethyl ethyl cellulose and combinations of two or more thereof. In one embodiment the cellulose derivative comprises microcrystalline cellulose, for example silicified microcrystalline cellulose. In one embodiment the microcrystalline cellulose is present extragranularly.

Suitably when present the cellulose derivative is present in an amount from 0.2% to 8%, for example from 1% to 15%, by weight of the composition. In one embodiment the cellulose derivative comprises microcrystalline cellulose present extragranularly in an amount from 0.2% to 8% by weight of the composition.

### Hydrophilic Colloid

A pharmaceutical composition according to the invention comprises a hydrophilic colloid, also known as a hydrocolloid. Suitably the hydrophilic colloid is present extragranularly in the composition. Examples of suitable hydrophilic colloids include alginic acid, carrageenan, gellan, pectin and agar. In one embodiment, the hydrophilic colloid is alginic acid.

Alginic acid, also referred to as algin or alginate, is a hydrophilic or anionic polysaccharide isolated from certain brown seaweed (Phaeophyceae) via alkaline extraction. It is present in cell walls of brown algae where it forms a viscous gel when binding with water. Alginic acid is a linear polymer consisted of L-glucuronic acid and D-mannuronic acid residues connected via 1,4-glycosidic linkages.

Suitably a hydrophilic colloid may be used in an amount from 0.1% to 10% by weight of the composition. In one embodiment, the hydrophilic colloid is alginic acid present in an amount from 0.7% to 5% by weight of the composition.

### Film Coat

When a composition according to the invention is in the form of a tablet, it may be coated or uncoated, In one embodiment a composition according to the invention is a film-coated tablet. Suitably the film coat is a transparent film coat e.g. a dye, although an opaque film coat e.g. as obtained when using a film coat in combination with an opacifier or a pigment such as titanium dioxide or a lake may also be used. For example one commercially available film coat is an Opadry coating system from Colorcon.

A film coat is typically used to enhance palatability and product appearance and to reduce friability and dusting of tablets. This is desirable for paracetamol tablets, as paracetamol is very bitter in taste and so may lead to low palatability and patient acceptance. However, the present inventors have found that aqueous film coating of tablets comprising aspirin can have a negative impact on the long-term storage stability of aspirin. Without being bound by any specific theory, it is believed that residual water, present in the film coating, and exposure to high temperatures during film forming and drying, can potentially accelerate hydrolytic cleavage of aspirin.

Notwithstanding this problem, a storage stable film-coated tablet according to the invention can be achieved by using a film-coat having a total build-up of no more than 2% by weight of the composition, for example from 0.3 % to 2% by weight of the composition, such as from 0.3% to 1.5% by weight of the composition. In one embodiment, the composition comprises a film-coated tablet wherein the amount of film coating is from 0.3 % to 1.5 % by weight of the composition.

In one aspect the coating time i.e. the time taken to apply and dry the film coat is controlled and kept to a minimum. For example for a composition wherein the total build of film coat is 0.3% or 2% by weight of the composition, the coating time is no longer than 15 minutes or 1 hour respectively.

### Disintegrating Agent

A pharmaceutical composition according to the invention may further comprise a disintegrating agent. When present, a disintegrating agent may be present intragranularly, extragranularly or both intragranularly and extragranularly. A disintegrating agent of use in the invention is suitably a conventional disintegrating agent, a super disintegrant or a combination of both. Super disintegrants represent a class of disintegrating agent which generally may be used in lower amounts in pharmaceutical compositions, as compared to conventional disintegrants.

Examples of suitable conventional disintegrating agents include starch, corn starch, pregelatinized starch, microcrystalline cellulose, celluloses and cellulose derivatives. A conventional disintegrating agent may be present in an amount ranging from 5% to 30% by weight of the composition, suitably from 8% to 20% by weight of the composition.

Examples of suitable super disintegrants comprise sodium starch glycolate, the sodium salt of carboxymethyl starch, modified cellulose such as the sodium salt of carboxymethyl cellulose, and cross-linked polyvinyl pyrrolidone such as crospovidone. In one embodiment, the disintegrating agent comprises a super disintegrant which is cross-linked polyvinyl pyrrolidone. In one embodiment the superdisintgrant is present intragranularly and extragranularly.

A super disintegrant may be used intragranularly in an amount ranging from 0.05% to 2% for example from 0.1% to 1% by weight of the composition. A super disintegrant may be used extragranularly in an amount from 0.05% to 2.0% for example from 0.5% to 1.5% by weight of the composition. In one embodiment, the super disintegrant is cross-linked polyvinyl pyrrolidone present intragranularly in an amount from 0.05% to 1% and present extragranularly in an amount from 0.05% to 1.5% by weight of the composition.

It is recognized in the art that some excipients are multifunctional. Therefore, the same excipient may perform more than one role in the composition, for example a given excipient may function both as a binding agent and as a disintegrating agent. For example, starches including pregelatinized starch can serve both as a binding agent and as a disintegrating agent. If a binding agent is incorporated in the composition that additionally has the functionality of a disintegrating agent, no additional disintegrating agent may need to be incorporated. Equally if a disintegrating agent is incorporated that additionally has the functionality of a binding agent, no additional binding agent may need to be incorporated. Accordingly, in one embodiment the disintegrating agent and the binding agent are the same excipient.

### Additional Components

Additional pharmaceutically acceptable components include a dye; colorant; flavorant; compression aid; preservative; wetting agent; bulking agent; adhesive; sweetening agent; lubricant such as magnesium stearate, calcium stearate, sodium stearate, stearic acid or talc; and a flow aid or glidant such as colloidal silicon dioxide (Cab-O-Sil, Syloid®). Suitably, when present, a lubricant or flow aid are each used in an amount ranging from 0.1% to 5.0% by weight of the composition. It is recognized that additional pharmaceutically acceptable components may be present as intragranular components as well as extragranular components.

Suitably, the intragranular components are mixed and subsequently granulated to form a granulate. Subsequently, the granulate is admixed with the extragranular components. In one embodiment the granulate comprises the following (%w/w of the total composition):
30% to 40% paracetamol
2% to 8% pregelatinized starch
0.05% to 0.2% povidone
2% to 6% calcium carbonate
0.1% to 1% crospovidone
q.s. water

In one embodiment the extragranular components comprise the following (%w/w of the total composition):
30 % to 40 % aspirin
5 % to 10 % pregelatinized starch
0.1 % to 2 % alginic acid
5 % to 10 % caffeine

Suitably a blend of aspirin, caffeine and pregelatinized starch may be further admixed with the remaining extragranular components and subsequently be admixed with the granulate. Alternatively, all extragranular components simultaneously are mixed with the granulate and subsequently compressed to form tablets, if that is the desired dosage form. In one embodiment, a composition to the invention is in the form of a tablet. In one embodiment, the composition is in the form of a monolayer swallow tablet.

The compositions of the invention may be prepared by tablet compression methods known in the art. For example, tablets can be prepared employing a tablet press fitted with an elongated shape tooling of about 17.5 x 7.5 mm, in accordance with the following specifications:

TABLE 1

Tablet compression specifications

| | |
|---|---|
| | Specification value |
| Weight (mg) | 730 mg ±18 mg |
| Hardness | 9-16 kp |
| Friability | Not more than 1% w/w |

The following examples are illustrations of the invention and should not be construed as limiting.

### Example 1

### API Excipient Compatibility Study

The compatibility of the active pharmaceutical ingredient (API) aspirin and the excipient calcium carbonate was evaluated with a drug excipient compatibility study.

### Particle Size Analysis

Three commercially available grades of calcium carbonate were used and their particle size distribution determined using a laser diffraction method. Different grades of calcium carbonate used for the experiments were Scoralite LL250 (available from Scora S.A.S, Rue de I′Usine, 62132 Caffiers, France), Sturcal M (available from Alpha House, Lawnswood Business Park, Redvers Close, Leeds, LS16 6QY) and CalEssence 450PCC (available from Specialty Minerals Inc., 35 Highland Avenue, Bethlehem, PA 18017, USA). The median diameter d50 of these different commercially available grades of calcium carbonate was determined with a Malvern Mastersizer 2000 particle size analyser employing the Hydro 2000S wet sample dispersion unit and the following parameters:

| | |
|---|---|
| Material | : Calcium Carbonate |
| Refractive Index | : 1.57 |
| Absorption | : 0.1 |
| Stir Speed | : 1400 Rotations per minute (RPM) |
| Dispersant | : Water |
| Dispersant refractive Index | : 1.33 |
| Obscuration | : 0.5 - 20 |
| Measurement Cycle | : 1 |
| Measurement Range | : 0.5 - 2000 µm |
| Sample size | : 0.5 mg |

The results of the particle size analysis are displayed in Table 2. d50 means the particle size distribution d50 value, also known as the median diameter or the medium value of the particle size distribution, which is the value of the particle diameter at 50 % in the cumulative volume distribution as determined with a particle size analyser with the laser diffraction method. d10 is the corresponding value at 10 % in the cumulative volume distribution and d90 is the corresponding value at 90 % of the cumulative volume distribution.

TABLE 2

Results the particle size analysis

| | | | |
|---|---|---|---|
| Grade of CaCO₃ | d10 | d50 | d90 |
| Scoralite LL250 | 2.910 µm | 16.799 µm | 39.709 µm |
| Sturcal M | 2.691 µm | 10.674 µm | 20.019 µm |
| CalEssence 450PCC | 1.002 µm | 5.281 µm | 12.114 µm |

### Sample Preparation

Samples were prepared by mixing of aspirin and calcium carbonate in a ratio of 1 : 0.5 by weight and transfer of defined amounts of the mixture into glass vials. The fine grade (CalEssence 450PCC) and the coarse grade (Scoralite LL250) of calcium carbonate were used, differing in median diameter (d50). This setup was used to determine the impact particle size distribution of calcium carbonate on the degradation of aspirin to salicylic acid. The aspirin used was of a coarse grade, the particle size was characterised by sifting through a sieve with the U.S. standard mesh number 30. Formulations F1, F2 and F3 were prepared having compositional details as provided for in Table 3. For each formulation, two samples were prepared one of which was closed with a lid whereas the other vial was left open. Formulation 1 was a control sample without any calcium carbonate.

TABLE 3

Compositional details of drug excipient compatibility study

| | Calcium carbonate (median diameter (d50)) | Grade of aspirin characterised U.S. standard mesh # |
|---|---|---|
| Formulation 1 (F1) (Control) | No calcium carbonate | 30 |
| Formulation 2 (F2) | 16.8 µm | 30 |
| Formulation 3 (F3) (Comparative) | 5.28 µm | 30 |

### Method

The samples were subjected to accelerated stability study conditions of 40° C. temperature and 75 % relative humidity (RH) for a study period of 30 days. On day 0 and on day 30, the content of salicylic acid in the samples was analysed. The salicylic acid content was put in relation to the initial amount of aspirin in the samples and presented in %, depicting the amount of aspirin that had degraded to salicylic acid during the study period.

### Results

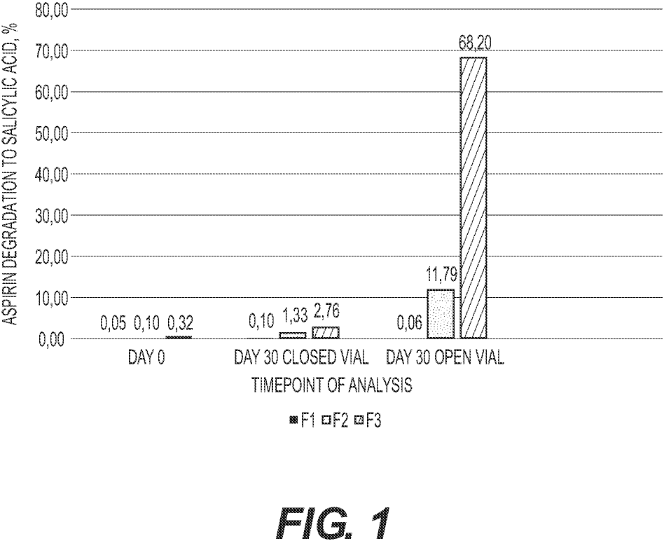

The results of this study are presented in Table 4 below and in FIG. 1.

TABLE 4

Results of API excipient compatibility study

| Formulation | % (w/w) of aspirin degraded to salicylic acid | | |
|---|---|---|---|
| | | Closed vial conditions | Open vial conditions |
| | Day 0 | Day 30 | Day 30 |
| F1 (Control) | 0.05 | 0.10 | 0.06 |
| F2 | 0.10 | 1.33 | 11.79 |
| F3 (Comparative) | 0.32 | 2.76 | 68.2 |

Samples of F2 and F3 showed enhanced degradation compared to the control samples of F1, illustrating the deleterious effect of calcium carbonate on aspirin stability. The hydrolysis of aspirin was further enhanced by storage under open vial conditions where the samples were subjected to 40° C. temperature and 75% relative humidity (RH). The comparison of the results of F3 and F2 revealed the significant influence of the average particle size of calcium carbonate. In the closed vial condition at 40° C. and 75% RH, in the sample of F3, twice as much aspirin degraded to salicylic acid during the study period of 30 days compared to the sample of F2. This difference was amplified in the open vial condition where 68% of the aspirin degraded to salicylic acid in the F3 sample which was more than six times the degradation apparent in the sample of F2.

### Example 2

### Stability Studies

Six batches of sample tablets were produced employing different average particle sizes of calcium carbonate and different mesh number of aspirin. These tablets were subjected to accelerated (40° C., 75 % RH) and long-term (25° C., 60% RH) stability testing conditions to evaluate the influence of the parameters on the degradation of aspirin to salicylic acid during the study period of 6 months.

### Preparation of Study Tablets

Batches of study tablets were produced from Formulations F4-F9 having the compositional details as provided for in Table 5 (compositional details) and Table 6 (average particle sizes of calcium carbonate and mesh number of aspirin) below:

TABLE 5

Compositional details of study tablets

| Ingredients | Formulations F4 - F9 (% w/w) | | | | | |
|---|---|---|---|---|---|---|
| | F4 | F5 | F6 | F7 | F8 | F9 |
| Paracetamol | 34.11 | 34.11 | 34.11 | 34.11 | 34.11 | 34.25 |
| Pregelatinized starch | 5.12 | 5.12 | 5.12 | 5.12 | 5.12 | 5.14 |
| Povidone | 0.17 | 0.17 | 0.17 | 0.17 | 0.17 | 0.17 |
| Calcium carbonate | 4.50 | 4.50 | 4.50 | 4.50 | 4.50 | 4.50 |
| Crospovidone | 0.40 | 0.40 | 0.40 | 0.40 | 0.40 | 0.40 |
| Alginic Acid | 1.02 | 1.02 | 1.02 | 1.02 | 1.02 | 1.03 |
| Colloidal Silicon Dioxide | 0.03 | 0.03 | 0.03 | 0.03 | 0.03 | 0.03 |
| Stearic Acid | 0.07 | 0.07 | 0.07 | 0.07 | 0.07 | 0.27 |
| Aspirin | 34.11 | 34.11 | 34.11 | 34.11 | 34.11 | 34.11 |
| Caffeine | 8.87 | 8.87 | 8.87 | 8.87 | 8.87 | 8.90 |
| Pregelatinized Starch | 6.14 | 7.50 | 7.50 | 6.41 | 6.41 | 6.04 |
| Crospovidone | - | - | - | 1.09 | 1.09 | 1.10 |
| Silicified Microcrystalline Cellulose | 2.18 | 3.00 | 3.00 | 3.00 | 3.00 | 3.01 |
| Silicon Dioxide | 2.46 | - | - | - | - | - |
| Stearic Acid | 0.82 | 1.09 | 1.09 | 1.09 | 1.09 | 0.89 |
| Total weight (%w/w) | 100.00 | 100.00 | 100.00 | 100.00 | 100.00 | 100.00 |
| Opadry II 57U18539* | 0.30 | 0.30 | 0.30 | 0.30 | 0.30 | - |
| Opadry II 57U510001* | - | - | - | - | - | 2.74 |
| Purified water | q.s. | q.s. | q.s. | q.s. | q.s. | q.s. |

* Coating weight gain (%) with respect to tablet core weight q.s. = quantum satis

TABLE 6

Grades of calcium carbonate and aspirin used in formulations F4-F9:

| | | |
|---|---|---|
| Formulation | Median diameter (d50) of calcium carbonate | Grade of aspirin characterised in U.S. standard mesh # / particle size distribution |
| Formulation 4 (F4) (Comparative) | 10.67 µm | 40/ at least 85% particles are in 105 µm-420 µm range |
| Formulation 5 (F5) (Comparative) | 5.28 µm | 30/ at least 60% particles are in 420 µm-850 µm range |
| Formulation 6 (F6) (Comparative) | 10.68 µm | 20/ at least 70% particles are in 180 µm-850 µm range) |
| Formulation 7 (F7), | 16.8 µm | 30/ at least 60% particles are in 420 µm-850 µm range |
| Formulation 8 (F8) | 16.8 µm | 40/ at least 85% particles are in 105 µm-420 µm range |
| Formulation 9 (F9) | 16.8 µm | 40/ at least 85% particles are in 105 µm-420 µm range |

The manufacturing process for the tablets of F4-F9 was as follows:

Part A: intragranular components, namely paracetamol, intragranular pregelatinised starch, Povidone-K25, calcium carbonate and crospovidone were sifted through a sieve and mixed in a rapid mixer granulator. To this mixture was added purified water quantum satis (q.s.) to form a granulate. The resulting granulate was wet milled, followed by drying in a fluid bed dryer. This dried granulate was sifted through a sieve and the sieve retention of oversized granules was milled, yielding a dried granulate of uniform granule size. This granulate was mixed with alginic acid and colloidal silicon dioxide and lubricated with stearic acid in a blender. The resulting part A blend was then transferred to a suitable blender.

Part B: Caffeine, extragranular pregelatinised starch and silicified microcrystalline cellulose were sifted and loaded onto the part A blend. Aspirin was sifted through a suitable sieve and loaded into the same blender. The ingredients were blended in the blender for a suitable time. Additional stearic acid was added for lubrication and the resulting mixture was then blended for a suitable time to form a master blend. Compression: The master blend was compressed to tablets of about 730 mg using caplet punches of about 17.5 × 7.5 mm.

Aqueous film coating: Resulting tablets (cores) were film coated in a suitable perforated coating pan with a film forming system as specified and purified water, up to a weight gain of the core of 0.3 % w/w or 2.74 % w/w, as specified.

Packaging: Coated tablets were packed in high density polyethylene (HDPE) bottles with a desiccant and firmly closed.

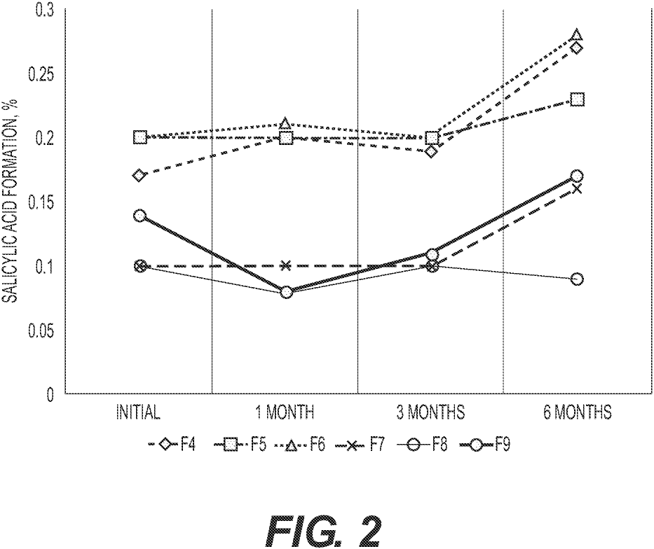

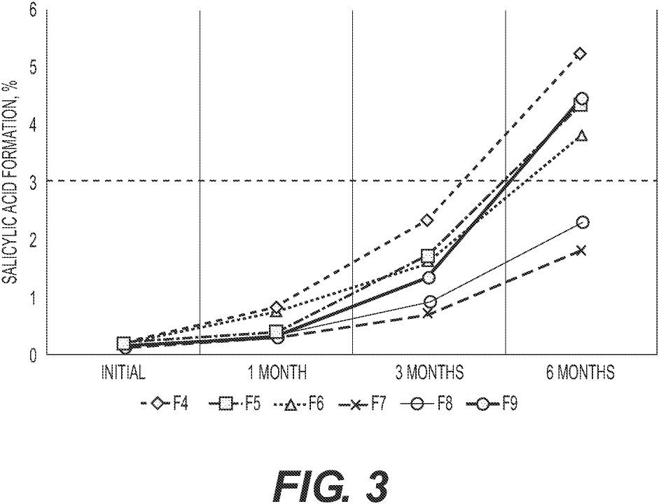

The resulting samples were subjected to accelerated and long-term stability study conditions for a study period of one, three and six months. The study conditions were 40° C. temperature and 75% RH for the accelerated study and 25° C. temperature and 60% RH for the long-term stability study. The salicylic acid content was analysed on day 0, after three months and after six months and expressed in relation to the initial content of aspirin in the samples and presented in percentage amounts, depicting the amount of aspirin that had degraded to salicylic acid during study period. The results of the study are shown in Table 7 and FIGS. 2 and 3.

### Results

TABLE 7

Results of stability studies

| Formulation | Salicylic acid content % | | | | | | |
|---|---|---|---|---|---|---|---|
| | | 25° C., 60% RH | | | 40° C., 75% RH | | |
| | Day 0 | 1 Month | 3 months | 6 Months | 1 months | 3 months | 6 months |
| F4 (Comparative) | 0.17 | 0.2 | 0.19 | 0.27 | 0.81 | 2.33 | 5.22 |
| F5 (Comparative) | 0.2 | 0.2 | 0.2 | 0.23 | 0.4 | 1.7 | 4.3 |
| F6 (Comparative) | 0.2. | 0.21 | 0.2 | 0.28 | 0.75 | 1.61 | 3.77 |
| F7 | 0.1 | 0.1 | 0.1 | 0.16 | 0.3 | 0.7 | 1.8 |
| F8 | 0.1 | 0.08 | 0.1 | 0.09 | 0.33 | 0.90 | 2.3 |
| F9 | 0.14 | 0.08 | 0.11 | 0.17 | 0.32 | 1.35 | 4.42 |

In FIG. 2, formulations comprising calcium carbonate with a d50 of 16.8 µm showed the lowest content of salicylic acid and therefore the most beneficial stability of aspirin upon all three timepoints of analysis. Salicylic acid content after six months was below the USP threshold of 3% for all formulations following storage at 25° C./ 60% RH.

In FIG. 3, upon accelerated stability study conditions, the three formulations comprising calcium carbonate of with a d50 of 10.67 µm or smaller exhibited an unsatisfactory salicylic acid content of greater than 3%. Surprisingly, the effect of different grades of aspirin could not outweigh the enhanced degradation of aspirin observed in formulations comprising calcium carbonate of a d50 of 10.67 µm or less. Formulations F7 and F8, comprising calcium carbonate of a d50 of 16.8 µm, showed a salicylic acid formation of less than 3% after six months. The results demonstrate that for a calcium carbonate and aspirin containing composition, stability of aspirin is surprisingly improved when formulated with calcium carbonate of a particular particle size.

Formulations F8 and F9 had the same composition of tablet core but differed in the amount of film coating applied to the core, F8 tablet cores were coated up to a weight gain of 0.3% by weight of the core, while F9 had tablet cores, coated up to 2.7 % by weight of the core. When subjected to accelerated stability condition for six months, F8 showed a salicylic acid level of less than 3% while formulation F9 exceeded the salicylic acid level beyond 3% (FIG. 3). Interestingly, F7 and F8 showed negligible aspirin degradation when subjected to long term stability conditions (FIG. 2). Without being bound to a specific theory, it is believed that diffused and trapped moisture in the core during the long aqueous coating process was responsible for degradation of aspirin, influence of this moisture was amplified under accelerated study conditions. This outcome lead to the observation that the amount of film coat on a core and the film coating process may also have an influence on the stability of aspirin.

### Example 3

### Dissolution Study

A dissolution study was conducted to evaluate the dissolution of the three APIs in the test formulation and compared with the dissolution with that of a marketed product.

### Sample Preparation

Test tablets were produced using Formulation F9 and a manufacturing process as described in Example 2. As control tablets, a commercially available product, marketed under the Name “Excedrin Extra Strength”, comprising the same amounts of each of paracetamol, aspirin and caffeine, but not comprising calcium carbonate or any basic agent was used.

The dissolution study was performed utilizing a USP it paddle apparatus rotating at 30 rpm, employing 900 ml of 0.05 M HCl at 37° C. as the dissolution medium. The percentage of dissolved API was determined at six minutes. This method and condition is considered to be discriminatory and predictive of the in vivo performance of the tested tablets. The study was conducted with one and with two tablets in the same amount of dissolution medium to evaluate dissolution in a half dose and single dose scenario. The difference in release between the test tablets and the control tablets was calculated by putting the amount of API released at six minutes from the test tablets in correlation to the amount of API released by the control tablets.

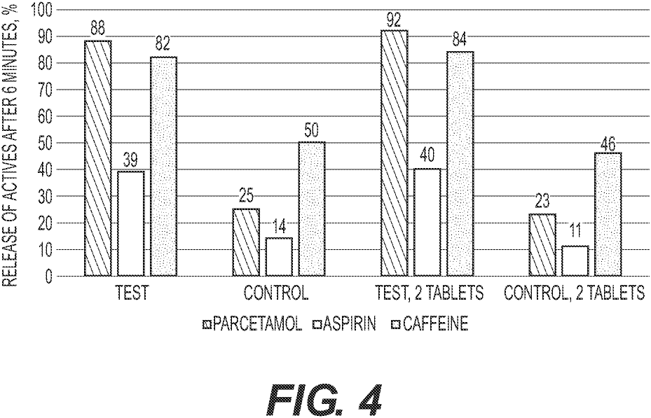

The results of the dissolution study are provided in Table 8 and FIG. 4 below.

TABLE 8

Results of dissolution study
Amount of API released from the tablet after six minutes, % (w/w)

| | Paracetamol | Aspirin | Caffeine |
|---|---|---|---|
| Dissolution of one tablet per dissolution vessel | | | |
| Test tablets (Formulation F9) | 88 | 39 | 82 |
| Control tablets (Excedrin extra Strength) | 25 | 14 | 50 |
| Difference in release, test / control | 3.5 | 2.8 | 1.6 |
| Dissolution of two tablets per dissolution vessel | | | |
| Test tablets (Formulation F9) | 92 | 40 | 84 |
| Control tablets (Excedrin extra Strength) | 23 | 11 | 46 |
| Difference in release, test / control | 4 | 3.6 | 1.8 |

The results show that Formulation F9 had superior dissolution characteristics compared to a currently marketed product comprising the same APIs and doses. The desired fast release of paracetamol was obtained. From the test tablets, 3.5 (one tablet, and 4 (two tablets) times as much paracetamol was released compared to the release from control tablets devoid of calcium carbonate. Surprisingly, the test Formulation F9 additionally showed enhanced release of aspirin and fast release of caffeine compared to the control formulation.

The significantly higher release of all three API at six minutes is expected to be predictive for a relatively fast drug release in vivo. F7 and F8 differ from F9 in neglectable adjustments of the amounts of some extragranular components and mainly in the amount of film coating, which was only 0.3% by weight of the core in F7 and F8 compared to 2.74% by weight of the core for F9, Therefore, formulations F7 and F8 are expected to exhibit similar fast dissolution, as observed with F9.

1. A pharmaceutical composition comprising intragranular and extragranular components wherein the intragranular components comprise paracetamol, calcium carbonate provided in the form of particles having a d50 greater than 11 µm, and at least one intragranular binding agent, and wherein the extragranular components comprise aspirin, and at least one hydrophilic colloid.

2. The pharmaceutical composition according to claim 1 wherein the composition further comprises a paracetamol adjuvant, present intragranularly and/or extragranularly.

3. The pharmaceutical composition according to claim 2 wherein the paracetamol adjuvant is present extragranularly.

4. The pharmaceutical composition according to claim 2 wherein the paracetamol adjuvant is present in an amount from 4% to 15% by weight of the composition.

5. The pharmaceutical composition according to claim 4 wherein the paracetamol adjuvant is caffeine.

6. The pharmaceutical composition according to claim 1 wherein the paracetamol is present in an amount from 20% to 50% by weight of the composition.

7. The pharmaceutical composition according to claim 1 wherein the paracetamol is present in an amount from 30% to 40% by weight of the composition.

8. The pharmaceutical composition according to claim 1 wherein the calcium carbonate has a d50 of from 11 µm to 30 µm.

9. The pharmaceutical composition according to claim 1 wherein the calcium carbonate has a d50 of from 15 µm to 25 µm.

10. The pharmaceutical composition according to claim 1 wherein the intragranular binding agent is a starch, a polymer, a cellulose derivative, or a combination of two or more thereof.

11. The pharmaceutical composition according to claim 10 wherein the starch is pregelatinized starch.

12. The pharmaceutical composition according to claim 11 wherein the pregelatinized starch is present in an amount from 1% to 20% by weight of the composition.

13. The pharmaceutical composition according to claim 1 wherein pregelatinized starch is present as the intragranular binding agent in an amount from 1% to 10% by weight of the composition and further present extragranularly in an amount from 1% to 10% by weight of the composition.

14. The pharmaceutical composition according to claim 10 wherein the polymer is a povidone, a polyvinyl alcohol, a polyethylene oxide, a poloxamer, a polymethacrylate, a carbomer, a polyethylene glycol, a calcium polycarbophil, or a combination of two or more thereof.

15. The composition according to claim 14 wherein the polymer is povidone present intragranularly in an amount from 0.01% to 5% by weight of the composition.

16. The composition according to claim 10 wherein the cellulose derivative is a hydroxypropyl cellulose, a hydroxypropyl methyl cellulose, a microcrystalline cellulose, a carboxymethylcellulose, a sodium carboxymethyl ethyl cellulose, or a combination of two or more thereof.

17. The pharmaceutical composition according to claim 16 wherein the cellulose derivative is microcrystalline cellulose present extragranularly in an amount from 0.2% to 8% by weight of the composition.

18. The pharmaceutical composition according to claim 1 wherein the aspirin is present in an amount from 20% to 50% by weight of the composition.

19. The pharmaceutical composition according to claim 1 wherein the aspirin is present in an amount from 30% to 40% by weight of the composition.

20. The pharmaceutical composition according to claim 1 wherein the aspirin is provided in particulate form and wherein at least 85% of the aspirin particles have a particle size distribution in the range 105 µm to 420 µm.

21. The pharmaceutical composition according to claim 1 wherein the hydrophilic colloid an alginic acid, a carrageenan, a gellan, a pectin, an agar, or a combination of two or more thereof.

22. The pharmaceutical composition according to claim 21 wherein the hydrophilic colloid is alginic acid present in an amount from 0.1% to 10% by weight of the composition.

23. The pharmaceutical composition according to claim 1 wherein the composition comprises a disintegrating agent.

24. The pharmaceutical composition according to claim 23 where the disintegrating agent comprises a super-disintegrant, the super-disintegrant being a cross-linked polyvinyl pyrrolidone, croscarmellose, a carboxymethyl cellulose, or a combination of two or more thereof.

25. The pharmaceutical composition according to claim 24 wherein the superdisintegrant is a cross-linked polyvinyl pyrrolidone present intragranularly in an amount from 0.05% to 1% by weight of the composition and extragranularly in an amount from 0.05% to 2.5% by weight of the composition.

26. The pharmaceutical composition according to claim 1 in the form of an uncoated tablet.

27. The pharmaceutical composition according to claim 1 in the form of a coated tablet.

28. The pharmaceutical composition according to claim 27 comprising a film coat in an amount from 0.3% to 1.5% by weight of the coated tablet.

29. The pharmaceutical composition according to claim 1 for use in the treatment of pain.
